(19) United States
(12) Patent Application Publication
(10) Pub. No.: US 2017/0361324 A1
(43) Pub. Date: Dec. 21, 2017

(54) VIEWER DEVICE AND METHODS FOR MAKING THE SAME

(71) Applicant: RAN Biotechnologies, Inc., Beverly, MA (US)

(72) Inventors: Roger A. Nassar, Swampscott, MA (US); Alexander D. Turland, North Reading, MA (US)

(21) Appl. No.: 15/628,513

(22) Filed: Jun. 20, 2017

Related U.S. Application Data
(60) Provisional application No. 62/352,128, filed on Jun. 20, 2016.
(60) Provisional application No. 62/361,748, filed on Jul. 13, 2016.
(60) Provisional application No. 62/426,909, filed on Nov. 28, 2016.

(51) Int. Cl. B01L 9/00; G01N 35/00; G01N 1/28; G01N 33/483

(52) U.S. Cl. CPC B01L 9/00; G01N 1/28; G01N 35/00722; B01L 2300/02; G01N 33/483

(57) ABSTRACT

A viewer device for observing and recording images of materials, such as chemical and biological materials is described. The viewer device includes a housing that provides a chamber for receiving a sample or a sample container that contains a sample of a material (e.g., chemical or biological material). The housing includes a viewing window for observing the sample, a port through which the sample or the sample container can be introduced to or removed from the chamber, and a coupling mechanism configured to removably and replaceably couple the housing to an observational device, such as a camera equipped device.

### PRIOR APPLICATIONS

This application claims the benefit of and priority to U.S. Provisional Application No. 62/352,128, filed on Jun. 20, 2016, U.S. Provisional Application No. 62/361,748, filed on Jul. 13, 2016, and U.S. Provisional Application No. 62/426,909, filed on Nov. 28, 2016, the entire teachings of each of which are incorporated herein by reference.

### FIELD

The present application relates generally to methods and apparatus for observing and recording images, and more particularly to methods and apparatus for observing and recording images of biological and chemical materials.

### BACKGROUND

Describing and recording observations and visual outcomes of experiments (e.g., chemical and biological experiments) is an essential part of conducting such experiments. One of the major advantages of these acts is allowing individuals, including those other than the operators, to re-visit these experiments and derive theories and conclusions without needing to be present at the time when the experiments are performed.

Experiments are often recorded in writing and, in some cases, using hand or software-drawn schemes. Photos obtained using cameras or other optical instruments can also be used to record experiments and their outcome. In fact, since portable cameras (e.g., portal digital cameras) are now widely available, particularly as part of portable electronic devices (e.g., smartphones, laptops, tablets, and wearable computer devices), photos obtained using such devices have become one of more frequently used means of recording observations during and/or at the conclusion of experiments.

However, images generated by many of such portable devices often lack the quality and/or reliability that is often needed for reproducing an experiment. These issues can also become more pronounced in cases where pictures are or may be an essential part of the experiment. For example, in cases in which comparison of images of a sample obtained at various stages of an experiment is required, factors such as inconsistent distances between the camera lens and the recorded items, inconsistent and/or poor lighting, inconsistent and/or poor focus, and variations in the background can cause difficulties in reproducing the experiment.

### SUMMARY

In one aspect, a viewer device according to the embodiments described herein can enable visual observation and recording of images related to materials, including but not limited to biologicals and chemicals, while controlling the image-taking process, the setting of the imaged item, and the background signals that could influence this process. As used herein, the term “image” is intended to include a still image or a video image.

In one aspect, a viewer device is disclosed, which includes a housing that provides a chamber for receiving a sample holder. The housing includes a viewing window for observing a sample contained in the sample holder when the sample holder is disposed in the chamber and a port through which the sample holder can be introduced into, and removed from, the chamber. The chamber can be configured for simultaneously receiving a plurality of sample holders.

In a related aspect, a system for analyzing a sample is disclosed, which includes a sample holder having at least one fluidic channel for receiving a sample, an input port for introducing the sample to said fluidic channel, and a viewer device. The viewer device comprises a housing that provides a chamber for receiving the sample holder. The housing comprises a viewing window for observing at least a portion of the sample flowing through the fluidic channel, a port through which the sample holder can be introduced into, and removed from, said chamber; and a coupling mechanism for removably and replaceably coupling said housing to an observational device such that the observational device can generate data via said viewing window about a sample flowing through said at least one fluidic channel.

In another related aspect, a viewer device having a housing that provides a chamber for receiving a sample holder is disclosed. The housing can include a viewing window for observing a sample contained in the sample holder when the sample holder is disposed in the chamber; a port through which the sample holder can be introduced into, and removed from, the chamber; and an observational device configured to generate data about the sample via the viewing window.

In other examples, any of the aspects above, or any system, method, apparatus described herein can include one or more of the following features.

The viewer device can further include a coupling mechanism for removably and replaceably coupling the housing to an observational device such that the observational device can generate data, e.g., image data, about the sample via the viewing window. The observational device can be a portable device. By way of example, the observational device can comprise a camera-equipped device for obtaining any of a picture and/or video of the sample. The observational device can comprise an analysis module for analyzing the data (e.g., image data) to generate information about the sample. By way of example, in some embodiments, the information can indicate a level of aggregation of at least one constituent of the sample. In some embodiments, the information can indicate color of at least a portion of said sample.

The camera-equipped device can be configured for acquiring visible images of a sample. Alternatively and/or additionally, the camera-equipped device can be configured for acquiring infrared and/or ultraviolet images of a sample. Further, the camera-equipped device can comprise any of a smartphone, a tablet, a laptop computer, a portable computer, or any device having an image acquisition mechanism included therein.

The coupling mechanism can comprise any of a clip, a railing, one or more clamps, or magnetic attachments, or vacuum cups and pads. The coupling mechanism can be configured to allow aligning a camera of the camera-equipped device with the viewing window of the viewer device. Further, the coupling mechanism can comprise at least one clip for removably and replaceably attaching the housing of the viewer device to an observational device, a railing to which the clip is movably coupled for adjusting the position of the viewer device relative to the observational device, and a knob coupled to the clip and the railing for securing the viewer device at a desired position along the railing. In some embodiment, the viewing window can comprise a camera and/or a lens, e.g., a magnifying lens that allows obtaining magnified images of a sample under observation.

The viewer device can include a light source coupled to the housing for illuminating the sample holder when disposed in the chamber. The light source can be, for example, a visible light source. At least a portion of the sample holder is substantially transparent to visible radiation to allow the light from the light source to illuminate at least a portion of a sample contained within the sample holder. Additionally or alternatively, the viewer device can comprise a light diffuser and/or a light filter that is optically coupled to the light source for diffusing and/or filtering the light emitted by the light source such that the diffused and/or filtered light illuminates the sample. The light source can comprise any of a light emitting diode (LED), a halogen lamp, a fluorescent, and a compact fluorescent light bulb. The light source can also include a color filter optically coupled to the light source for selecting a desired color for illuminating the sample.

The viewer device can also include a locking mechanism that is coupled to the housing for engaging with at least a portion of the sample holder so as to inhibit movement of said sample holder in said chamber. By way of example, the locking mechanism can comprise a lip disposed proximate to the port, through which a sample holder can be introduced into the viewer device's chamber, for engaging with a proximal end of a sample holder disposed in the chamber so as to inhibit at least one of up-down movement and rotation of the sample holder.

The viewer device can also comprise a mechanism coupled to the housing for manipulating the sample holder disposed in the chamber. The mechanism for manipulating the sample holder can be configured to cause any of physical shaking, rotation, spinning, vortexing, and reversing the orientation of the sample holder disposed in the chamber. By way of example, the mechanism for manipulating the sample holder to re-suspend the sample therein can comprise at least one spring disposed in said chamber for mechanical coupling to said sample holder such that pulling up the sample holder at least partially from the chamber and releasing it can cause agitation of a sample disposed in the sample holder. Alternatively or additionally, the sample can be manipulated by applying a magnetic field arranged to attract magnetic particles of the sample to a one or more directions, partially or totally isolating these magnetic particles from the rest of the sample.

In some embodiments, the chamber of the viewer device can be configured for simultaneously receiving a plurality of sample holders.

In some embodiments, the viewer device can further comprise a heater coupled to the housing for heating a sample contained in the sample holder. Alternatively or additionally, a cooler can be coupled to the housing for cooling a sample contained in the sample holder. The sample holder can comprise any of a test tube, a filter, or a slide, a cuvette, a fluidic channel, a line of continuously flowing fluid or any holder composed of any material that allows light to traverse through the sample and/or reach the sample. Alternatively or additionally, the sample holder can comprise at least one fluidic channel for receiving a sample. In some such embodiments, the viewer device allows obtaining an image, e.g., a video image, of a sample as it flows through the fluidic channel. Further, embodiments disclosed herein are not limited to the use of a sample holder. The sample can be disposed in the viewing field of the viewer device via any suitable technique known in the art. For example, the sample can be placed directly into the viewer device for observation and recording. In some embodiments, the sample can be any object suitable for observation with the viewer device, for example, an insect, a coin, a gem, a pearl, a stamp, a plant, a section of a plant, a mineral, or a hair, which is directly placed within the viewing field of the viewer device (e.g., in the viewer device) for observation.

In a related aspect, a viewer device is disclosed, which includes a housing providing a chamber for receiving a sample holder, where the housing comprises a viewing window for observing a sample contained in said sample holder when the sample holder is disposed in said chamber, and a port through which the sample holder can be introduced into, and removed from, said chamber. A camera is coupled to the housing and is configured to obtain images of said sample via said viewing window. In some embodiments, the camera can be integrated within the housing of the viewer device.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention.

FIG. 1A illustrates an example of a viewer device according to some embodiments disclosed herein.

FIG. 1B illustrates another example of a viewer device according to some embodiments disclosed herein.

FIG. 2 illustrates an example of components that can be included in a viewer device according to some embodiments disclosed herein.

FIG. 3A illustrates an example of a viewer device, according to some embodiments disclosed herein, which includes a mechanism for enabling attachment to camera-equipped devices.

FIG. 3B illustrates an example of some of the components that can be included in a viewer device according to some embodiments disclosed herein.

FIG. 3C is an illustrative example of a viewer device having components such as those shown in FIG. 3B.

FIG. 3D illustrates a snapping mechanism that can be used with a viewer device according to certain embodiments disclosed herein.

FIG. 3E illustrates an example agitation mechanism that can be used with a viewer device according to certain embodiments disclosed herein.

FIGS. 3F-3G are illustrative examples of a viewer device, according to the embodiments disclosed herein, in which the viewer device includes a locking mechanism for securing the sample container.

FIGS. 4A and 4B are illustrative examples of some of the application of the viewer device disclosed herein.

FIGS. 5A and 5B are illustrative examples of some of the other application of the viewer device disclosed herein.

FIGS. 6A-6F illustrate images of an example material that can be observed by a viewer device in accordance with some embodiments described herein.

FIG. 7A illustrates an example of an attachment mechanism that can be used with a viewer device in accordance with some embodiments disclosed herein.

FIG. 7B illustrates another example of an attachment mechanism that can be used with a viewer device in accordance with some embodiments disclosed herein.

FIG. 7C illustrates yet another example of an attachment mechanism that can be used with a viewer device in accordance with some embodiments disclosed herein.

FIG. 7D illustrates another example of an attachment mechanism that can be used with a viewer device in accordance with some embodiments disclosed herein.

FIG. 7E illustrates yet another example of an attachment mechanism that can be used with a viewer device in accordance with some embodiments disclosed herein.

FIG. 7F illustrates another example of a viewer device according to some embodiments disclosed herein.

FIG. 8A illustrates another example of the viewer device in accordance with one or more embodiments disclosed herein.

FIG. 8B is an example of a fluidic chip that can be used with the embodiments disclosed herein.

FIG. 9A illustrates a viewer device according to some embodiments disclosed herein.

FIG. 9B illustrates an example of the viewer device of FIG. 9A with a rack that includes one or more observation samples.

FIG. 9C illustrates an example of the rack shown in FIG. 9B.

FIG. 9D illustrates another example of a viewer device according to some embodiments disclosed herein.

FIG. 9E illustrates yet another example of a viewer device according to some embodiments disclosed herein.

FIG. 9F illustrates another example of a viewer device according to certain embodiments disclosed herein.

FIG. 9G illustrates yet another example of a viewer device according to certain embodiments disclosed herein.

FIGS. 10A-10C illustrate an example embodiment of the viewer device that can be used for observing and/or recording dynamic samples.

FIG. 11 is an illustrative embodiment of a viewer device according to the embodiments disclosed herein.

FIG. 12 is a high-level block diagram of a system for observing and recording experiments according to some embodiments disclosed herein.

### DETAILED DESCRIPTION

A viewer device according to the embodiments disclosed herein can be used for visual observation and recording of experiments. For example, the viewer device can be used to obtain images from the materials (e.g., biologicals and chemicals) used or obtained in various experiments. The viewer device can be configured to control the image-taking process by, for example, controlling the arrangement and setting of the items being imaged and/or controlling the background signals that can influence the image taking process. Furthermore, the viewer device can be configured to manipulate the items being imaged, for example by physical shaking, rotation, spinning, vortexing, reversing orientation of the sample holder, or application of a magnetic field.

The term “image,” as used herein, is intended to include both still and video images. The term “about,” as used herein to modify a numerical value, is intended to indicate a variation of at most 5% around the numerical value.

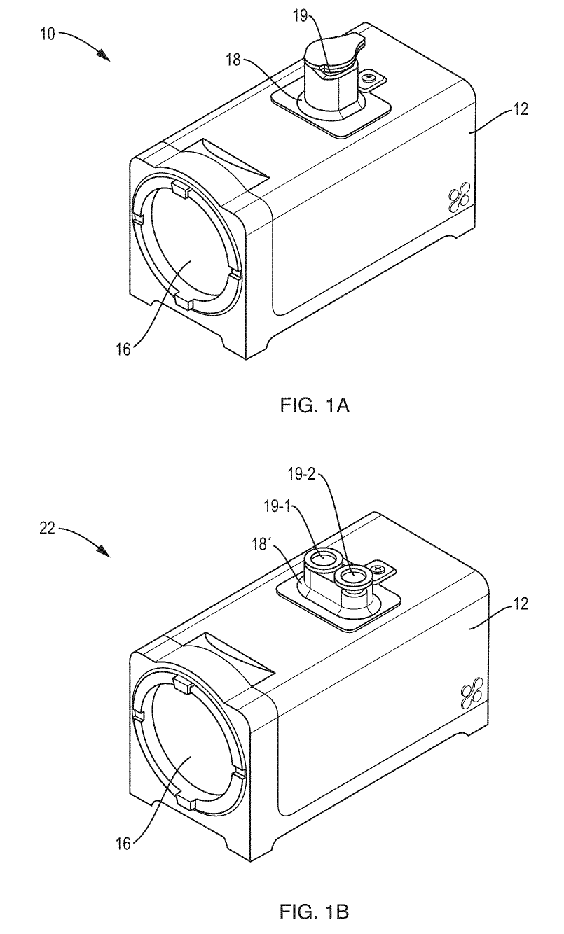

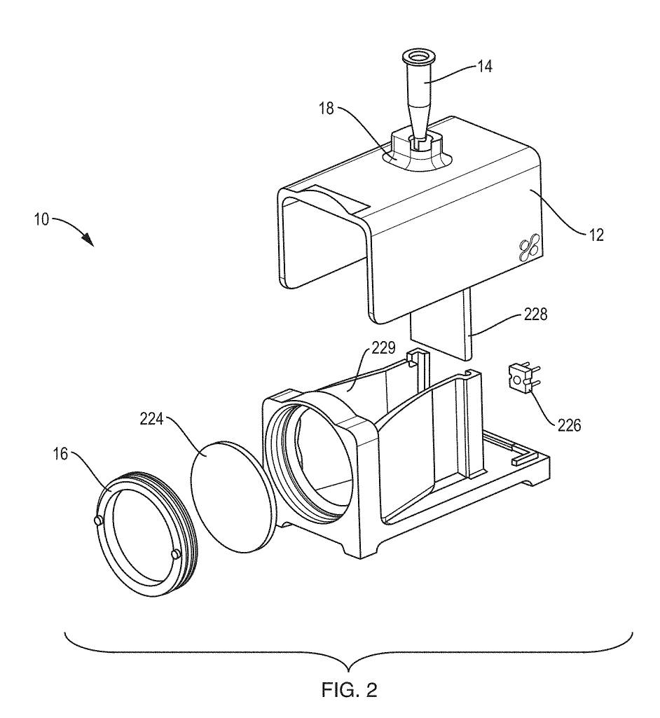

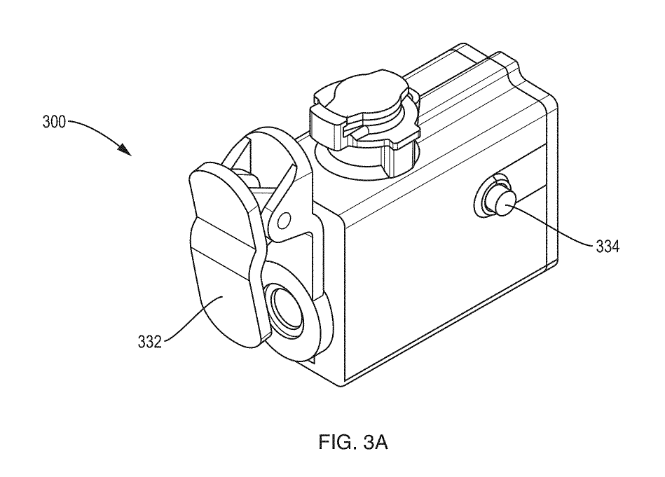

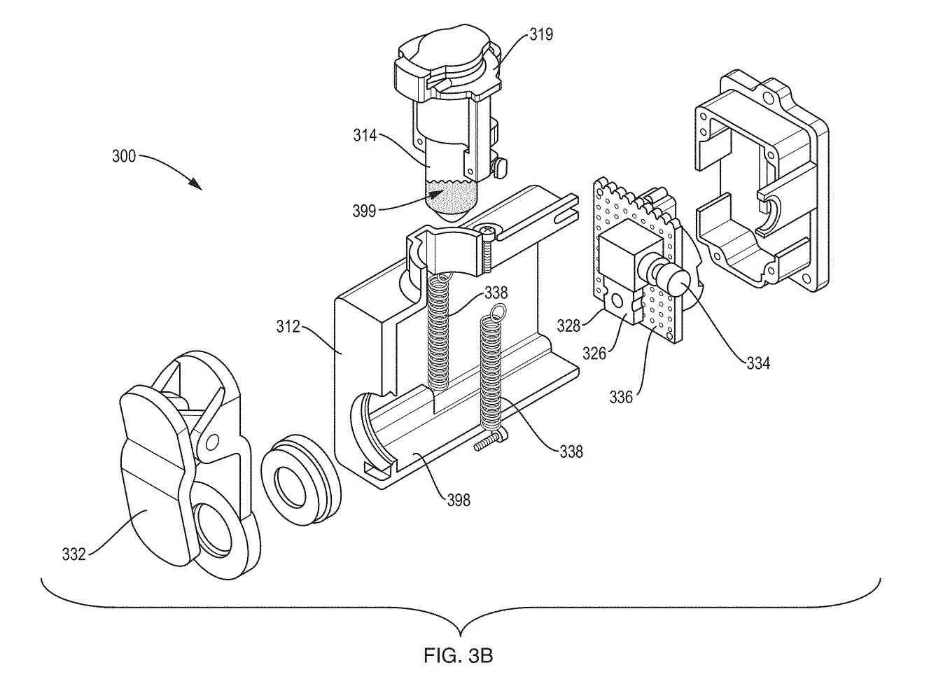

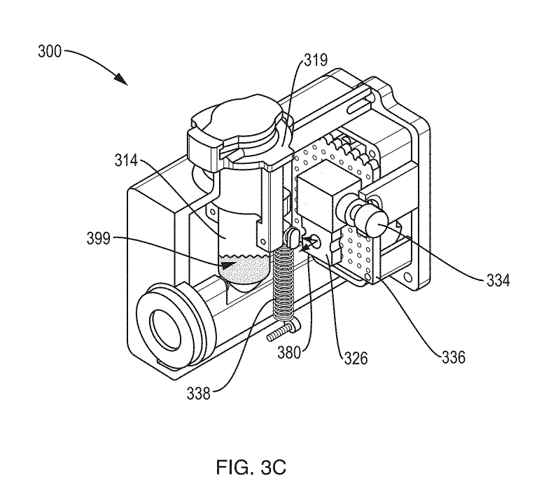

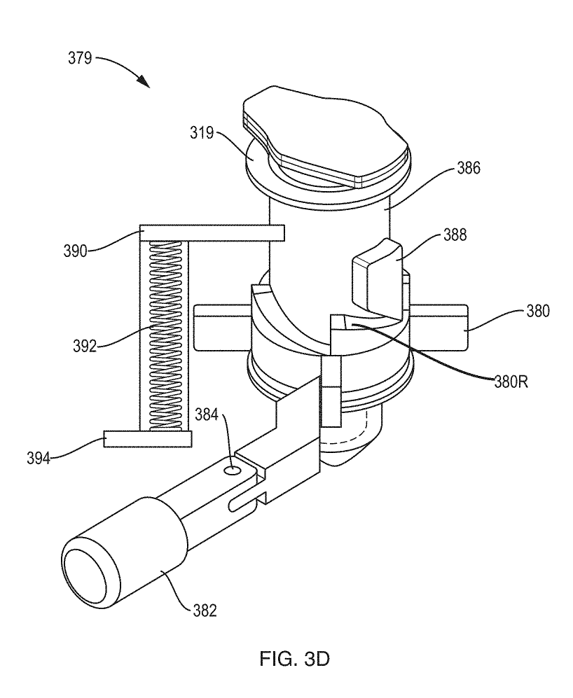

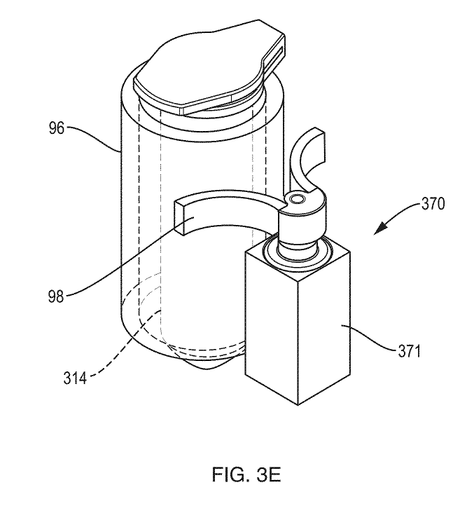

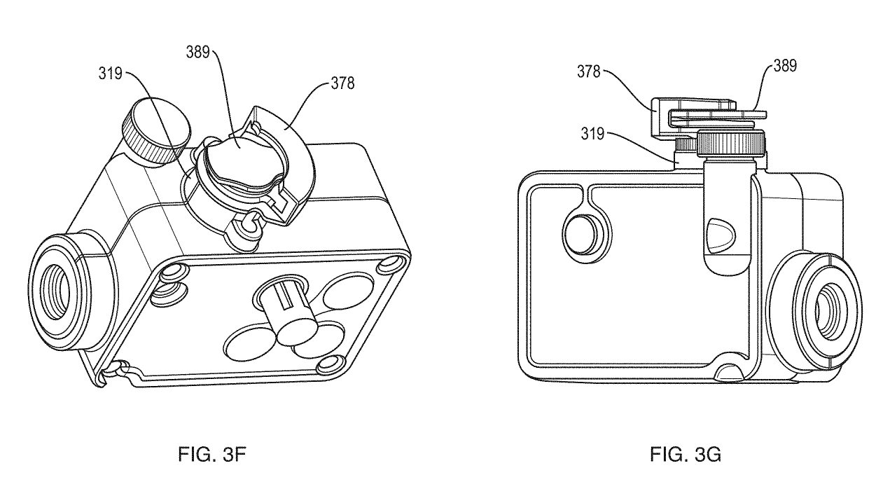

FIG. 1A is an example of a viewer device 10 according to some embodiments disclosed herein. The viewer device 10 can include a housing 12 having a chamber (e.g., chamber 229 shown in FIG. 2) for receiving a sample (not shown, see e.g., sample 399 shown in FIG. 3B) of a material (e.g., chemical, biological, or other item), which hereinafter is generally referenced as “sample” or “observation sample.” The housing 12 can also include a viewing window 16, through which the sample can be observed, e.g., viewed. The sample can be inserted or removed from the chamber and device 10 through a port 18, which can be configured to receive one or more sample holders or samples. Generally, the port 18 can assume any shape or size suitable for an intended application. For example, in the embodiment shown in FIG. 1A, the port 18 is configured to receive a cylindrical container that holds the sample (e.g., sample container 14 such as test tube, shown in FIG. 2, hereinafter “sample container”).

The chamber 229 and the viewing window 16 can have any size or shape suitable for an intended application. For example, the chamber 229 and the viewing window 16 can be arranged to have a variable/adjustable size and/or shape. Specifically, the chamber 229 and the viewing window 16 can be arranged such that the size and shape of the chamber 229 and/or viewing window 16 can be varied depending on the size of the item to be examined. Furthermore, these components can vary depending on the application in which the device is being used. For example, if the application requires simultaneous examination of two samples, side by side, chamber 229, viewing window 16 can be configured to accommodate such setup. Furthermore, in implementations in which the window 16 houses one or more magnifying lenses, the strength of this magnification can imply the geometry and position of window 16 in relation to chamber 229 and components associated with this chamber. Further, for example, in embodiments that use a cylindrical sample holder, such as a test tube, the size and shape of the chamber 229 and/or viewing window 16 can be selected or adjusted, e.g., widened or narrowed, depending on the size and/or shape of the cylindrical sample holder (e.g., test tube) being used. The sample/experiment can be viewed, observed, and/or recorded via the viewing window 16 using any suitable technique known in the art. For example, the sample can be viewed, observed, and/or recorded by an observer (e.g., human observer, not shown) that observes and records the experiment. Alternatively or additionally, as described in further details below, the sample/experiment can be viewed, observed, and/or recorded via the viewing window 16 via an observational device (not shown), such as a camera.

In some embodiments, the port 18 can have a fixed size that is selected to allow the insertion of one or more sample holders into the chamber of the viewer device. Additionally or alternatively, the port 18 can have a variable/adjustable size and/or shape. Specifically, the port 18 can be arranged such that its shape and/or size can be selected or adjusted (e.g., widened or narrowed) to accommodate one or more sample holders being inserted into or removed from the chamber 229.

As noted above, the port 18 can be configured to receive one or more sample holders. For example, in the embodiment illustrated in FIG. 1A, the port 18 includes one receptacle 19 for receiving and/or removing a sample holder. However, the port 18 can be configured to accommodate simultaneous insertion and/or removal of more than one sample holder. FIG. 1B illustrates an embodiment of the viewer device 22 in which the port 18′ is configured to receive more than one sample holder. Specifically, in this example, the port 18′ includes two receptacles 19-1, 19-2, through each of which a sample holder can be inserted or removed from the chamber of the viewer device. The port(s) can have any other suitable configuration. For example, as described in further details below, the ports can have various geometries and can be arranged to accommodate items and sample containers having conforming shapes and/or structures. For example, the port(s) can be configured to accommodate sample holders, such as test tubes, filters, slides, etc.

FIG. 2 illustrates an example of components that can be included in a viewer device 10 according to some embodiments disclosed herein. As shown in FIG. 2, the viewer device 10 can include a housing 12 that surrounds a chamber 229 in which an observation sample, e.g., contained in a sample holder, can be placed. The housing 12 can also include a viewing window 16. The size and/or shape of the viewing window 16 can be selected or adjusted to accommodate the size/shape of the observation sample and/or the sample container 14.

Further, as shown in FIG. 2, in this embodiment, the viewing window 16 can accommodate a magnifying lens 224. The magnifying lens can be any suitable magnifying lens available in the art. The size and magnifying power of the magnifying lens 224 can be varied depending on the observation sample and /or the application.

The viewer device 10 can also include a light source 226, a light filter (not shown), and a light diffuser 228. The light source 226, light filter, and the light diffuser 228 can be any suitable light source, light filter, or light diffuser available in the art. For example, the light source 226 can comprise at least one of an LED light source, a halogen light source, a fluorescent light source, a compact fluorescent light source, or one or more colored light bulbs.

The light source 226, the light filter, and the light diffuser 228 can be adjustable. For example, the power and type of the light source can also be adjustable depending on the observation sample and/or the application at hand. Further, the light source 226 can be configured to emit light at various wavelengths, e.g., at visible, such as wavelengths in a range of about 400 nm to about 700 nm, and infrared wavelengths, such as in a range of about 750 to 1200 nm. The wavelength of the light source 226 can be selected and or be adjustable depending on the observation sample, the application, and the required range of wavelengths for that application. Similarly, the type and light filtering or diffusing characteristics of the light filter or light diffuser 228 can be adjustable depending various factors, such as the required light output.

FIG. 3A illustrates an example of a viewer device 300, according to some embodiments disclosed herein, which includes a mechanism 332 (hereinafter “attachment mechanism”) for enabling attachment of the viewer device to one or more camera-equipped devices. The attachment mechanism 332 can be any suitable attachment mechanism. For example, the attachment mechanism 332 can be at least one of a clip, a railing, one or more clamps, or a combination thereof.

In the example shown in FIG. 3A, the attachment mechanism 332 comprises a clip 332 that can be configured to removably and replaceably attach the viewer device 300 to another device (not shown). The other device can be any suitable device known in the art. For example, the other device can be a camera equipped device (not shown), such as a smartphone, a laptop, a tablet, or a wearable computer device.

The viewer device 300 can also comprise one or more button(s) or switch(es) 334, which can be configured to control one or more functions of the viewer device 300, upon activation or deactivation. The button(s) or switch(es) 334 can control various functions within the device. For example, the button(s) or switch(es) 334 can be configured for use in activating or deactivating the light source (e.g., by turning on and off the light source 226, shown in FIG. 2), activating or deactivating one or more components that can cause a physical shaking motion of a sample holder disposed in the chamber of the viewer device, activating or deactivating one or more components that can cause the rotation of a sample holder disposed in the viewer device, and/or controlling various other actions and movements of the observation sample and its sample container.

FIG. 3B illustrates an example of some of the components that can be included in a viewer device 300 according to some embodiments disclosed herein. As noted above, the viewer device 300 can include a light source 326 and a light diffuser 328. The light source 326 and the light diffuser 328 can be any suitable light source or light diffuser available in the art. Further, the light source 326 and the light diffuser 328 can be adjustable such that their functions and outputs can be adjusted based on various factors, including the features of the observation sample 399 and the observation container 314. The viewer device 300 can also include one or more switches 334 for controlling various functions of the viewer device 300.

The light source 326 and one or more switches 334 (e.g., for controlling the light source 326) can be mounted on a circuit board 336. Further, the viewer device 300 can include one or more (e.g., a pair) of springs 338. The springs 338 can be configured to bias a sample holder 314 against the floor 398 of the viewer device's housing 312, and facilitate the removal of the sample holder from the chamber, when desired. The springs 338 can further be configured such that they can be controlled by one or more switches similar to switch 334. For example, the springs 338 can be configured such that they are engaged, for example in response to the insertion of the sample container 314 into the port 319 of the viewer device 300. Once engaged, the springs 338 can facilitate maintaining the sample container 314 in place, while the sample 399 is being observed and recorded. As described later, with reference to FIG. 3D, the springs 338 can further be configured such that they can be deactivated to release (e.g., by pushing up, away from the floor 398 of the housing 312) the sample container 314. The activation and deactivation of the springs 338 can be performed using any suitable technique known in the art, for example using a switch 334, or by exerting a mechanical force (e.g., by pushing down the sample container 314), or a combination thereof.

It should be noted that the pulling up and release actions on the sample container 314 can cause agitation of a sample 399 or sample holder 319, e.g., a solution containing chemicals and biologicals, disposed in the container 314. Such agitation can, for example, result in re-suspending deposited materials at the bottom of the observation sample 399. Further, the agitation of the sample 399 can be used for mixing multiple phases of materials contained in the sample holder.

FIG. 3C is an illustrative example of a viewer device having components such as those shown in FIG. 3B. As shown, the spring 338 can be used to secure the sample container 314 for the duration of observation and recording. As noted, one or more switches 334 can be coupled to various components of the viewer device 300 and used to control the functions of the viewer device 300. For example, as shown in FIG. 3C, the switch 334 can be coupled to the light source 326 and be used to control the light source 326, for example by turning on or off the light source 326. The light source 326, upon activation, can emit light (generally shown by arrows 380) onto the sample 399 included in the container. The springs 338 and the switch 334 can further activate or deactivate functions that result in focusing the emitted light 380 onto the sample 399 in the sample container 314. For example, the switch 334 can activate a motor (not shown) that moves the light source 326 away or towards the sample container 314. Alternatively or additionally, the switch 334 can be used to manually (e.g., by using a switch as a lever or handle) move the light source 326 (or the circuit board 336 upon which the light source 326 can be mounted) towards or away from the sample container 314. It should be noted that although described as moving the light source 314 towards and away from the sample source, in some embodiments the light source 314 can move in any direction or orientation in the three dimensions, for example by being moved up or down and/or by being titled in various directions and orientations.

As noted above, in certain embodiments, the viewer device can include one or more features, such as a snapping mechanism, configured to cause agitation of the sample container 314. FIG. 3D is an illustrative example of a snapping action mechanism 379 that can be used with a viewer device 300, e.g., the viewer device illustrated later in FIG. 3F or FIG. 3G. according to certain embodiments disclosed herein. As shown in FIG. 3D, the snapping action mechanism 379 can include an indexed snap toothed wheel 380 and a button 382 configured to perform a vertical tube-snapping action. Further, the structure 379 can include a shaft 386 in which a sample holder can be lodged. As shown, the shaft 386 can be connected to the port 319 through which a sample holder can be introduced into the lumen of the shaft 386.

The structure 379 can also include an elbow 388 that is firmly attached to the shaft 386 or is part of the structure of shaft 386. The shaft 386 can be firmly connected to one end of a spring 392 via a connection 390. The other end of the spring 392 can be connected to a fixed surface via the connection 394.

The structure 379 can be configured such that activation of the button 382 (e.g., when the button 382 is depressed), results in engaging and spinning the toothed wheel 380. While wheel 380 is spinning, the elbow 388 travels up on one of the ramps 380R of wheel 380. As the elbow 388 is traveling up the ramp of the wheel 380, the shaft 386 moves upward and results in stretching the spring 392 out of its original position. After the elbow 388 reaches the top of the ramp 380R, it can drop down suddenly due to the return of the spring 392 to its original position, thereby causing the shaft 386 to move downward in a vertical snapping action. Such snapping action can cause agitation of the sample inside the sample holder. Upon release of button 382, the button 382 can flex out of the way around a hinge 384 and return to its original position and ready to engage another tooth of the wheel.

FIG. 3E is an illustrative example of an automated agitation mechanism 370 that can be used with the embodiments described herein. The agitation mechanism 370 can include a motor 371 and one or more arms 98 connected to the motor 371. The agitation mechanism 370 can be disposed within the viewer device such that the one or more arms 98 of the agitation mechanism 370 are adjacent to the observation sample container 314 and can come in contact with the sample container 314, as the arms 98 are oscillated back-and-forth by the motor. The motor 371 can be connected to one or more switches or knobs that activate the motor 371. Generally, the motor 371 can be any suitable motor available in the art and can be activated via any suitable means known and available in the art. The motor 371, upon activation, can cause the arms 98 to move (e.g., horizontally, in a back and forth motion or in circular motion) such that they come periodically in contact with observation sample container 314 and cause the observation sample container 314 to shake. The contact between the arms 98 and the sample container 314 can be, for example, in the form of a repeated slapping motion that causes agitation of the sample 399 in the sample container 314. The motor 371 can be configured to allow for adjustment of the degree of the agitation of the sample holder, and consequently a sample contained in the sample holder, caused by the back-and-forth motion of the arms 98. For example, the motor 371 can be configured to move the arms at a faster/slow or more/less powerful rate depending on the amount of agitation required.

Further, the viewer device 300 can also include one or more features for securely maintaining the observation container in the viewer device 300. For example, as shown in FIGS. 3F-3G, the viewer device 300 can include a lip 378 that can be used to securely hold the sample container within the viewer device. In some embodiments, the lip 378 can be configured to lock the sample container in one or more specific positions within the viewer device (e.g., a position optimal for observing and/or recording the experiment).

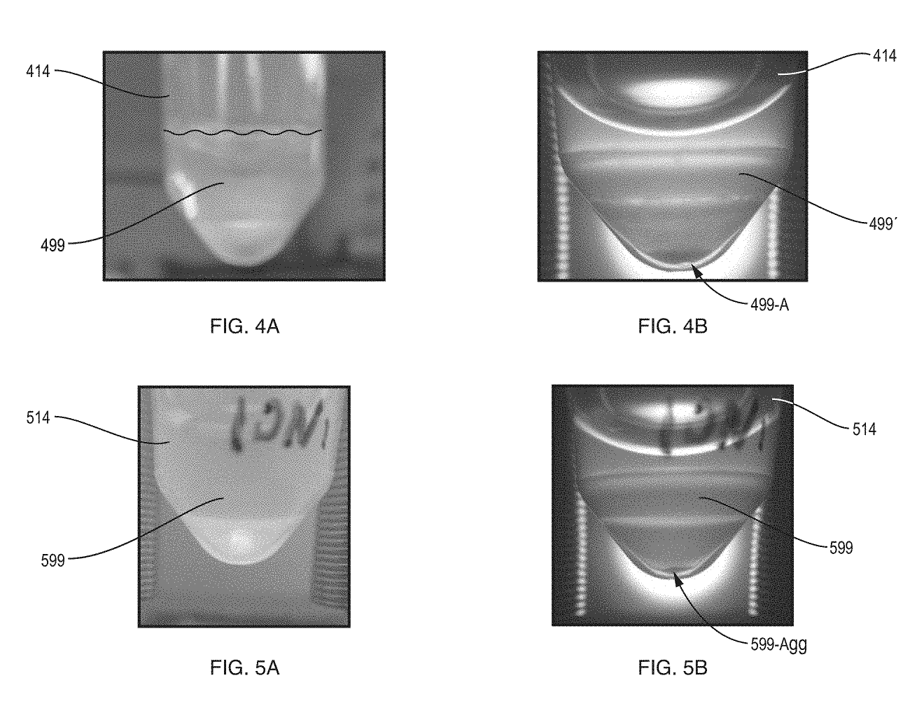

FIGS. 4A and 4B are illustrative examples of some of the application of the viewer device disclosed herein. As shown in FIG. 4A, aggregated materials at the bottom of a observation sample 499 (e.g., materials in a solution contained in the observation container 414) can be difficult to observe if the observation is recorded (e.g., by obtaining an image) under normal conditions (e.g., if the image of the sample is obtained while holding container 414 in front of a camera, as shown in FIG. 4A). However, as shown in FIG. 4B, the presence of the aggregated materials 499-A can be clearly observed at the bottom of the tube. An image obtained from the sample 499′ can clearly illustrate the presence of the aggregated materials 499-A in the observation sample 499′. Therefore, inserting the container 414, and consequently that of the sample 499 disposed in the container, into the device 300 and collecting an image using the device can improve the quality of the resulting image and allow for accurate analysis of the observed materials 499-A.

The viewer device can further facilitate and improve the recordation and analysis of a sample by directing appropriate frequencies and intensities of light waves at the sample. FIGS. 5A and 5B illustrate an example of some of the other applications of the viewer device in which application of appropriate amount of diffused waves allows for observation and recordation of aggregated materials within a sample, which would otherwise not be easily observable under normal light conditions.

Specifically, as shown in FIG. 5A, aggregated materials included in the bottom of an observation sample 599 cannot be visually observed under normal lighting conditions. However, as shown in FIG. 5B, upon application of diffused light (e.g., generated using light diffuser 228, shown in FIG. 2), the aggregated materials 599-Agg can readily be observed at the bottom of the solution container 514.

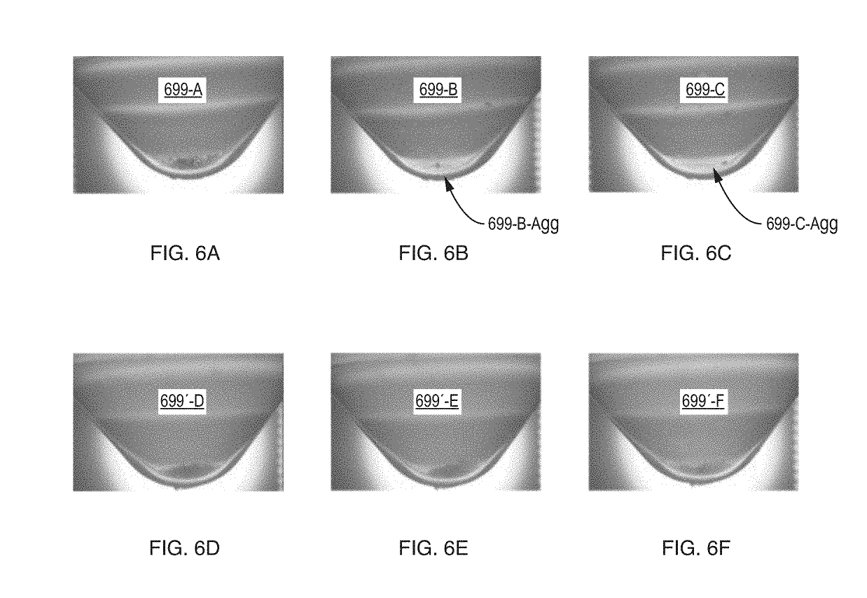

FIGS. 6A-6F illustrate images of an example of a material that can be observed by a viewer device in accordance with some embodiments described herein. Specifically, FIGS. 6A-6C illustrate an observation sample containing 0 cells/mL of Escherishia coli, while FIGS. 6D-6F illustrate an observation sample containing 10⁷ cells/mL of Escherishia coli. FIGS. 6A and 6D were obtained at 0 resuspension, FIGS. 6B and 6E were obtained at 1 resuspensions, and FIGS. 6C and 6F were obtained at 2 resuspensions. These examples are presented to demonstrate the potential benefits of agitation and/or resuspension of a sample (e.g., using an resuspension mechanism, such as that shown in FIG. 3D) prior to observing and recording images of a sample.

As shown in FIG. 6A, at 0 resuspension mark, there is very little observable difference between the solution 499-A containing 0 Escherishia coli cells/mL and a solution containing 10⁷ cells/mL of Escherishia coli cells/mL. When the solution is re-suspended once (one resuspension, shown in FIGS. 6B and 6E), the aggregates 699-B-Agg in the solution containing 0 Escherishia coli cells/mL (FIG. 6B) were re-suspended, while the aggregates in the solution containing 10⁷ Escherishia coli cells/mL were not re-suspended. Additional resuspension(s) can further illustrate the difference between the solutions. For example, as shown in FIGS. 6C and 6F, after 2 resuspensions, the aggregates 699-C-Agg in the solution containing 0 Escherishia coli cells/mL are re-suspended and are clearly visible in a photo obtained from the sample 699-C, while the aggregates in the solution containing 10⁷ Escherishia coli cells/mL (FIG. 6F) are not re-suspended.

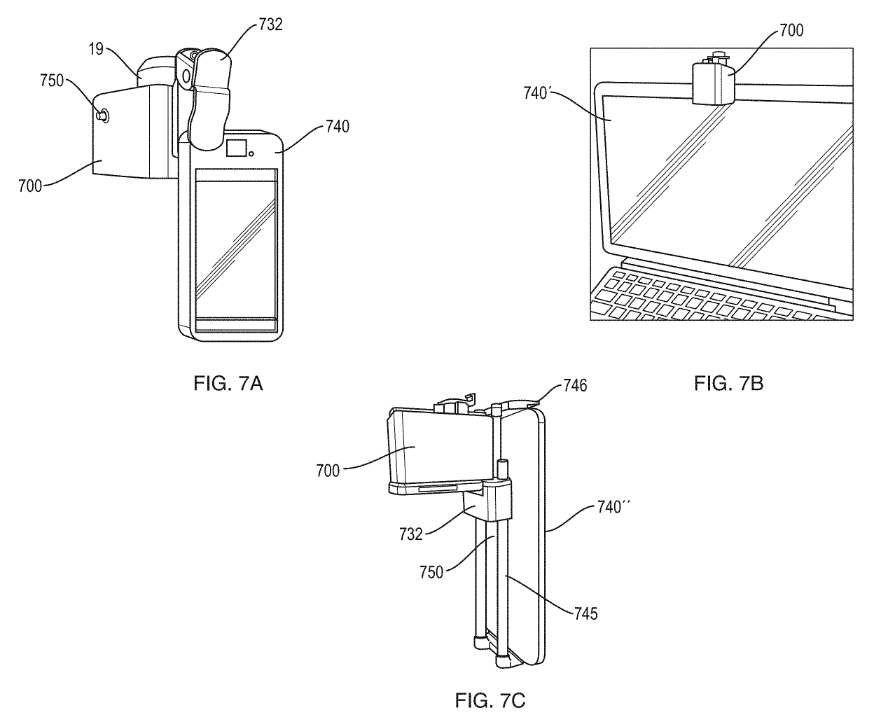

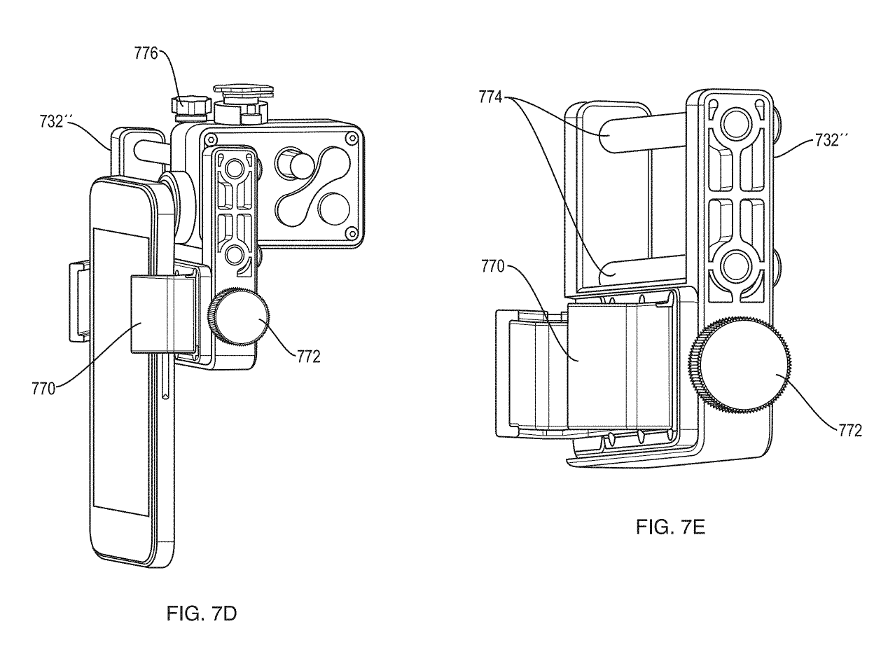

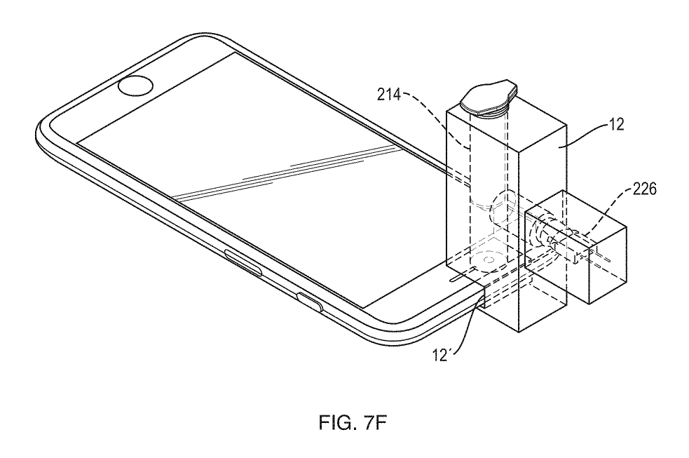

FIG. 7A illustrates an example of an attachment mechanism that can be used with a viewer device 700 in accordance with some embodiments disclosed herein. In the example shown in FIG. 7A, the viewer device 700 is attached to a camera-equipped device 740 (e.g., a digital camera or a mobile phone that includes a built-in digital camera) using a clip-shaped attachment mechanism 732. As noted previously, with respect to FIG. 3B, the attachment mechanism 732 can be any suitable attachment mechanism. For example, the attachment mechanism 732 can be a clamp or a clip, magnetic attachments, or vacuum cups and pads.

The viewer device 700 can be attached to the camera-equipped device 740, using the attachment mechanism 732, in a manner that the lens of the camera (not shown, e.g., the built-in camera of the mobile phone) is aligned with the viewing window (not shown here, see e.g., viewing window 16, shown in FIG. 1) of the viewer device 700. A sample holder (not shown here, see e.g., FIG. 3B) containing a sample can be introduced into the chamber of the viewer device via the port 19, and camera-equipped device 740 can then be used for obtaining photos/images of the sample. Light emitted by a light source and/or diffused by a light diffuser (not shown here, see e.g., FIG. 3B) can be used to enhance and improve the quality of the images acquired by the mobile phone and allow for enhanced recordation of the sample.

FIG. 7B illustrates another example of the viewer device in accordance with some embodiments disclosed herein. In this example, a laptop computer is used as the camera-equipped device 740′ and a viewer device according to an embodiment is coupled to the laptop computer via a clip. As noted above, embodiments presented herein are not limited to the use of any specific camera-equipped device. Any portable or stationary camera-equipped device can be used. Further, as described below in more details, in some embodiments, the viewer device 700 can include a built-in camera that can be used for obtaining images of samples. In such embodiments, the use of a camera-equipped device is no longer required and the viewer device 700 can be used as a standalone device for obtaining images of experiments.

FIG. 7C illustrates yet another example of a viewer device 700 in accordance with some embodiments disclosed herein. As noted above, the attachment mechanism 732 can include a variety of different coupling mechanisms. In the example shown in FIG. 7C, the attachment mechanism 732 can comprise a railing 745 that can allow the positioning of the viewer device 700 at various vertical positions with respect to the camera-equipped device 740″. The railing 745 can be adjustable and have one or more features that allow for coupling of the attachment mechanism 732 to the camera-equipped device 700. For example, as shown in FIG. 7C, the railing can be coupled to one or more end pieces 745, 746 that are configured to hold the camera-equipped instrument 740″ in place. The one or more end pieces 745, 746 can be any suitable structure and can assume any suitable size or form that allows the end pieces to facilitate attachment of the viewer device to the camera-equipped instrument. For example, as shown in FIGS. 7D-7E, the attachment mechanism 732″ can comprise one or more side clamps 770. The side clamps 770 can be adjustability controlled and secured by a knob 772. The attachment mechanism 732″ can also comprise a railing 774 that can be used to position the viewer device. Further, a knob 776 can adjustability control and secure the viewer device to the railing 774. In some embodiments, the knob 776 can be used to move the viewer device and/or the camera equipped device with respect to one another. In use, the viewer device can be moved up and down along the railing so as to align the viewing window of the viewer device with the camera of the camera-equipped device. Once a proper position is found, the knob 772 can be used to fix the position of the viewer device relative to the camera-equipped device.

The configuration and arrangement of the components included in the viewer device is not limited to the configurations and arrangements illustrated herein. For example, as shown in FIG. 7F, the body 12 of the viewer device can be configured to allow for vertical positioning of the sample holder 214 above the lens of the camera-equipped device. Specifically, in this embodiment, the body 12 of the viewer device can include a notch 12′ into which a portion of the camera-equipped device (a mobile phone in this embodiment) can be inserted such that the camera lens will be positioned below the sample holder. The bottom of the sample holder can be transparent to visible radiation so as to allow the camera to obtain, via a viewing window positioned below the chamber into which a sample holder can be disposed, image(s) of a sample contained in the sample holder. Further, although the light source 226 is shown as being placed on the side of the sample container 214, the light source 226 can be positioned at any position or orientation with respect to the sample container 214. Furthermore, interchangeable positioning (not shown here) of container 214 within the same body 12 can allow vertical, horizontal, or any other positioning of the container 214 in relation to the light source 226 or the viewing window 16.

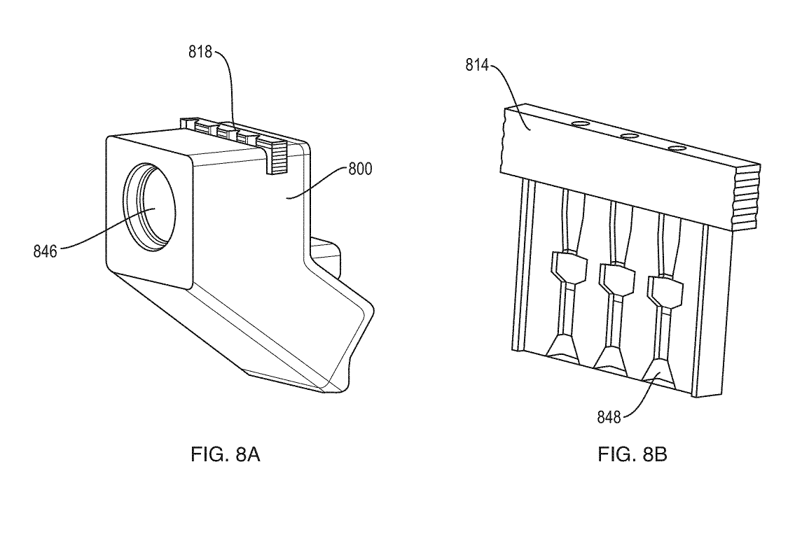

Further, as noted above, the observation sample container can assume any suitable shape, size, or feature. For example, as shown in FIGS. 8A-8B, in some embodiments, the viewer device 800 can receive a sample holder 814, which is in the form of a fluidic (e.g., microfluidic) chip. The microfluidic chip 814 can include one or more slots 848 for receiving one or more observation samples (not shown).

Furthermore, as noted above, the port of the viewer device can also assume any suitable shape and size available in the art and can be adjustable to securely receive the observation sample. For example, as shown in FIG. 8A, the port 818 of the viewer device 800 can be configured such that it can receive the fluidic (e.g., microfluidic) chip 814 containing the observation sample.

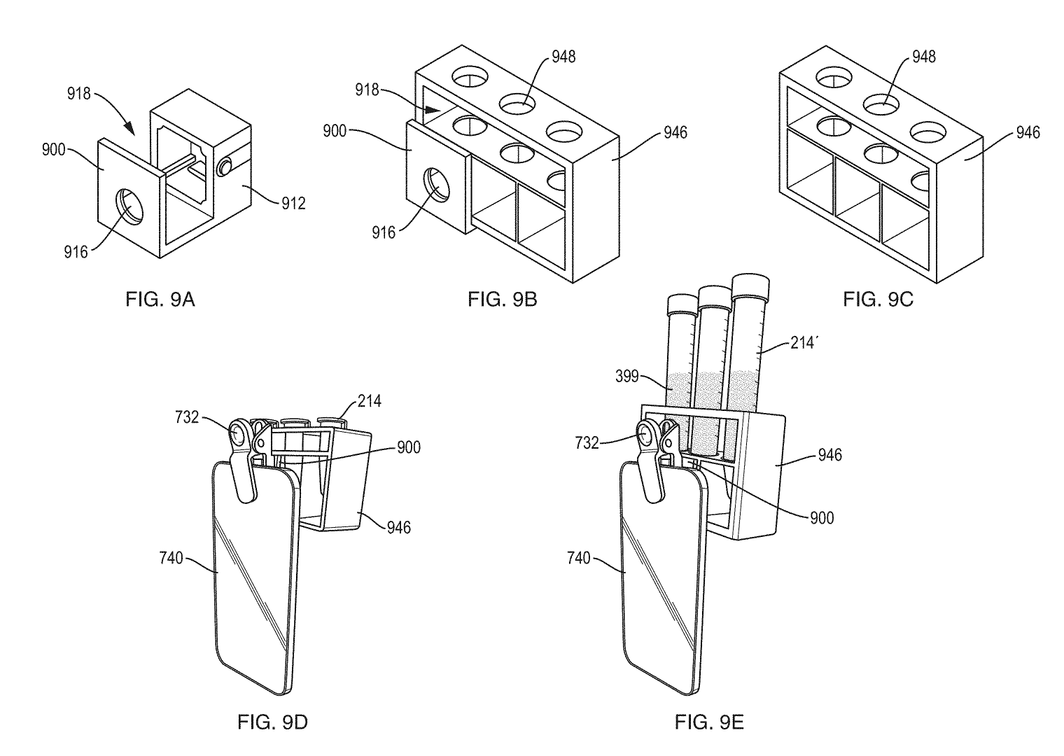

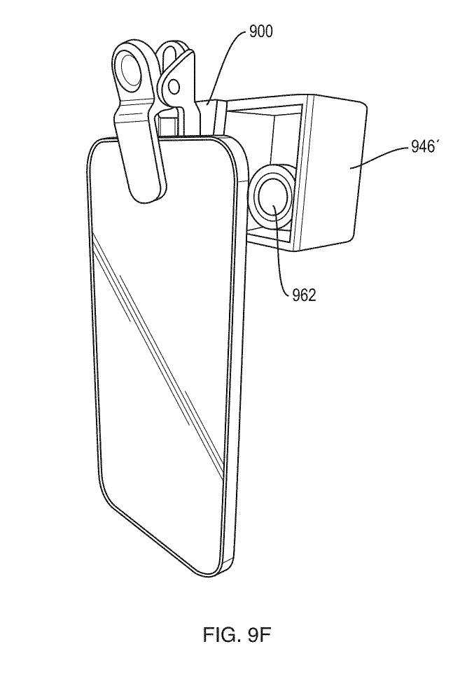

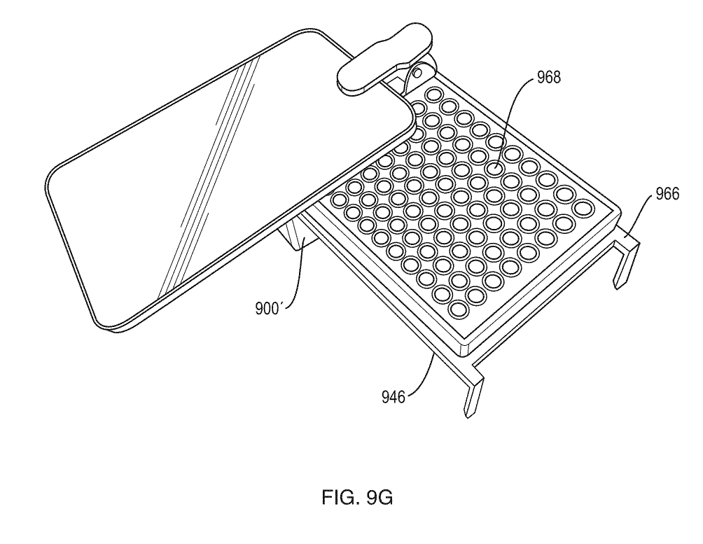

FIG. 9A is an example of a viewer device 900 according to certain embodiments disclosed herein that utilizes a port 918 configured to receive various sample containers. Specifically, as shown in FIG. 9A, the housing 912 can be configured to include a port 918 that can accommodate a variety of interchangeable structures and observation sample containers. FIGS. 9B-9C illustrate some examples of the structures that can be used with the viewer device 900. As shown in FIG. 9B, the port 918 can be configured to receive a rack 946 having one or more openings 948 for receiving one or more sample containers. For example, the rack 946 can include one or more circular-shaped openings 948 configured to receive one or more cylindrical sample containers, such as one or more test tubes. In the example shown in FIG. 9B, the rack 946 is shown after it has been inserted into the port 918 of the viewer device. The rack 946 can be movable within the port 918 and configured such that a desired sample container, from among the sample containers included in the rack 946, can be selected for observation by moving the rack 918 within the port 918 and placing the desired container within the viewing range of the viewing window 916.

FIG. 9C illustrates an example of a rack 946 that can be used with the viewer device 900 according to an embodiment of the present teachings. As shown, the rack 946 can include one or more openings 948 for receiving one or more sample containers. For example, as shown in FIG. 9D, the viewer device 900, which is coupled to a camera-equipped device 740, can include a rack 946 capable of holding three 2-milliliter observation sample containers 14. In this example, the rack 946 is configured to hold three observation sample containers. The desired sample container 214 (the container holding the sample being observed and recorded) can be placed within the viewing range of the viewing window 916 such that an image of the sample can be obtained using the camera-equipped device 740 and the viewer device 900.

Further, the rack 946 can be adjustable or configurable to accommodate sample containers having various shapes and sizes. For example, as shown in FIG. 9E, the rack 946 can be capable of holding 10-milliliter tubes that contain the observation sample 399.

Generally, racks 946 having any suitable shapes and sizes can be used. The size and shape of the rack 946 used for observation and recordation can depend on the size and shape of the sample container which contains the observation sample. For example, as shown in FIG. 9F, a rack 946 capable of holding one or more filters 962 can be utilized. The filters 962 can be any suitable filter cartridge known in the art. For example, the filters 962 can be at least one of a syringe filter or a wheeler filter. Generally, the rack 946 can be configured to hold one or more filters. For example, such setup can be used to observe samples that have been filtered out of solutions. Specifically, the filtered samples deposit on the surfaces of filters 962. Filters 962 can then be inserted into rack 946, rack 946′ can then be attached to device 900, which can allow observation and recording of the filtered samples. In some embodiments, a filter can be used to filter one or more particles (e.g., insoluble particles) of the sample. The filtered particles can remain on the filter or filter membrane (e.g., on the surface of the filter), and the viewer device can be used to view, observe, record, and/or analyze the filtered particles.

The rack 946 can also be adjustable to accommodate well-plates of different sizes and shapes. For example, as shown in FIG. 9G, the rack 946 can be configured to hold objects, such as a multi-well plate 968. The multi-well plate 968 can be any multi-well plate available in the art. The multi-well plate 968 can include one or more wells capable of receiving an observation sample. The rack 946 can be mounted onto a railing or stage 966 and configured such that it allows for the movement of the viewer device 900′, the rack 946, or both. Specifically, the rack 946 can be configured to allow the viewer device 900 to be positioned at different locations in order to record images of each and all of the wells of the multi-well plate 968.

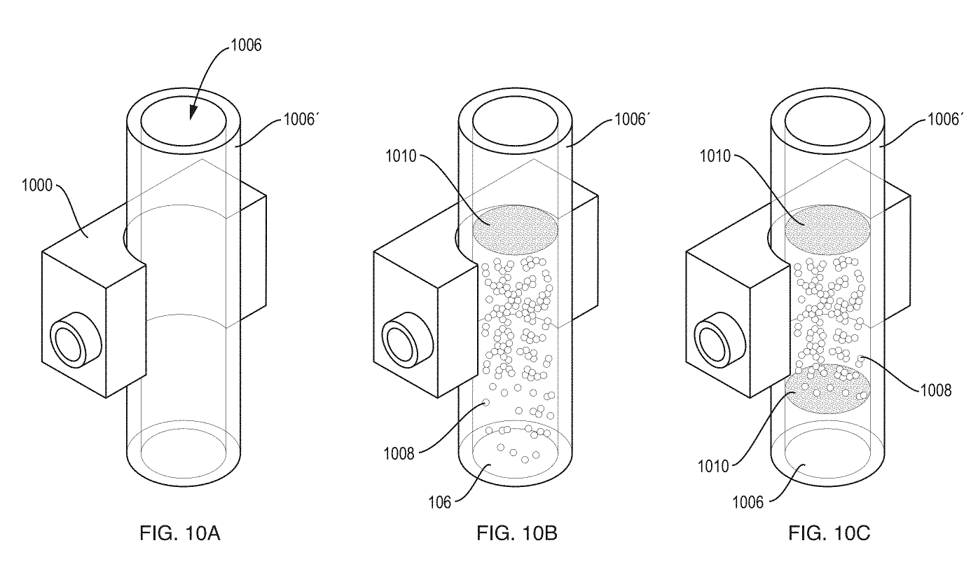

As noted above, embodiments disclosed herein are not limited to obtaining still/static images of observation samples and/or observing static samples. FIGS. 10A-10C illustrate an example embodiment in which the viewer device 1000 can be utilized to observe and record a dynamic observation sample. Specifically, the viewer device 1000 can be configured to adapt to an in-line setup in order to observe dynamic samples where, for example, fluids are flowing through a channel 1006 of a sample holder 1006′. As shown in FIG. 10B, viewing particles 1008 can be filtered out of a flowing fluid through a filter 1010. Alternatively or additionally, as shown in FIG. 10C, viewing particles 1008 can be held between two filters 1010. The sample holder can be placed within a chamber of the viewer device such that the particles flowing through the channel 1006 can be observed and image(s) thereof can be obtained. The device 1000 can help with observation of fluids as they flow through channels 1006. If the objective is to filter out and remove particles 1008 from the fluids passing through 1006, the device 1000 can help visualize what and how much is being filtered, as depicted in FIG. 10B. If the objective is to capture materials on resins in a chromatography-like setup, then device 1000 helps observe changes in the resins 1008 trapped between two filters 1010 in FIG. 10C.

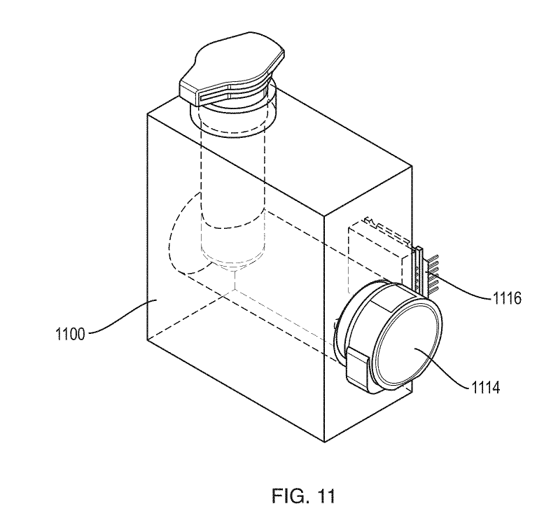

As noted previously, in addition to or in place of utilizing a camera equipped device for observing and/or recording images of samples, the viewer device can include a built-in camera. FIG. 11 is an illustrative example of a viewer device 1100 that utilizes a built-in camera 1114. The camera 1114 can be any suitable camera available in the art. For example, the camera 1114 can be a digital camera. Further, the device 1100 can include one or more features that allow for storage and/or transfer of images obtained using the camera 1114. For example, the device 1100 can include one or more adaptors 1116 for wireless fidelity (wifi) and or short-range wireless interconnection of devices (Bluetooth). Generally, any suitable feature known in the art for obtaining and/or transferring images can be used with the embodiments presented herein. Further, the viewer device 1100 can be coupled with any suitable feature in the art that facilitates observing and recording of a sample.

For example, a viewer device having a built-in camera and/or the camera-equipped device (e.g., a smart phone or laptop) to which the viewer device is attached can include and/or be coupled to one or more application software configured to store and process images obtained using the viewer device. Examples of such processing can include interpreting recorded images by analyzing the colors, shapes, sizes, or responses of features in the recorded images to generate digital information.

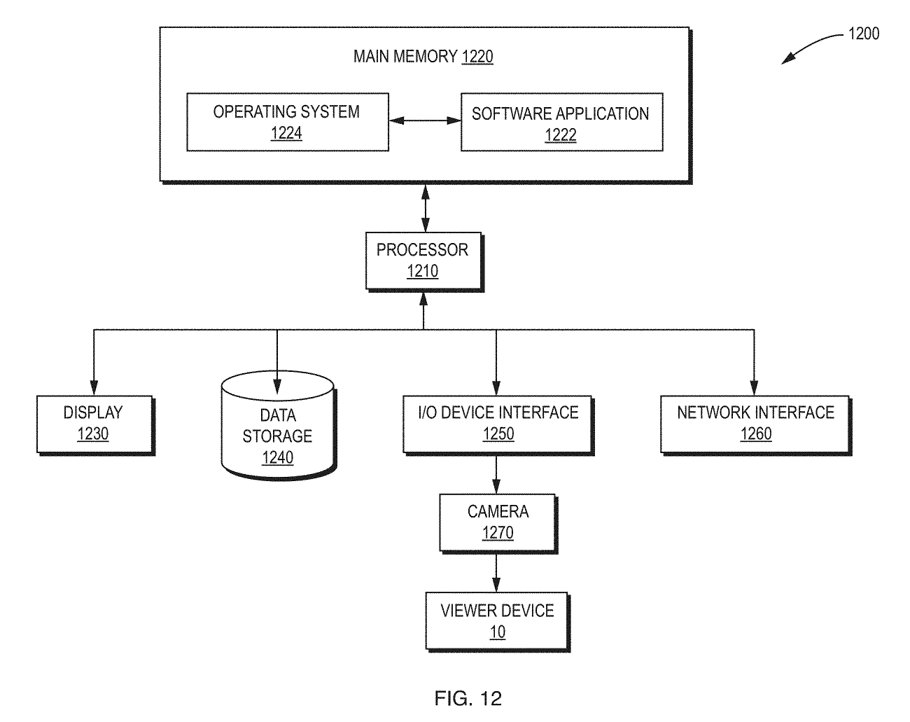

FIG. 12 is a high-level block diagram of a system 1200 for observing and recording samples according to some embodiments disclosed herein, which can be implemented in viewer devices according to various embodiments. The system 1200 can be implemented using a camera-equipped device coupled to the viewer device and/or using a viewer device that includes a built-in camera. As described in further details below, the system 1200 can be coupled to a processor (e.g., processor included in the camera-equipped device, processor 1210 included in a viewer device having a built-in camera, processor coupled with a camera-equipped device or with a viewer device having a built-in camera, etc.) that carries out some of the functions described herein. Generally the functions disclosed herein can be carried out and implemented by any suitable computer system and/or in digital circuitry or computer hardware.

The processor 1210 can implement the various functions and methods described herein. For example, the processor 1210 can implement application software and procedures that obtain and record images of an observation sample using the viewer device 10. The processor 1210 can be connected to the main memory 1220. The processor 1210 and the main memory 1220 can be included in or supplemented by special purpose logic circuitry.

The processor 1210 can include a central processing unit (CPU, not shown) that includes processing circuitry configured to manipulate instructions received from the main memory 1220 and execute various instructions. For example, the processor 1210 can be a general and/or special purpose microprocessor and any one or more processors of any kind of digital computer. Generally, the processor 1210 can be configured to receive instructions and data from the main memory 1220 (e.g., a read-only memory or a random access memory or both) and execute the instructions. The instructions and other data can be stored in the main memory 1220.

As shown in FIG. 12, the main memory 1220 can include an operating system 1224. The main memory 1220 and the operating system 1224 can be configured to implement various operating system functions. For example, the operating system 1224 can be responsible for controlling access to various devices, memory management, and/or implementing various functions of the camera-equipped device and/or the viewer device (which can include a built in camera). The main memory 1220 can be any form of non-volatile memory included in machine-readable storage devices suitable for embodying data and computer program instructions. For example, the main memory 1220 can be magnetic disk (e.g., internal or removable disks), magneto-optical disks, one or more of a semiconductor memory device (e.g., EPROM or EEPROM), flash memory, CD-ROM, and/or DVD-ROM disks.

The main memory 1220 can also hold application software 1222. For example, the main memory 1220 and application software 1222 can include various computer executable instructions, application software, and data structures such as computer executable instructions and data structures that implement various aspects of the embodiments described herein. For example, the application software 1222 can include various computer executable instructions, application software, and data structures such as computer executable instructions and data structures that can be used to observe and record experiments.

The main memory 1220 can also be connected to a cache unit (not shown) configured to store copies of the data from the most frequently used main memory 1220. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The functions performed by the camera-equipped device and the viewer device, such as observing and recording experiments (e.g., by taking still or dynamic images of the samples) can be implemented in digital electronic circuitry or in computer hardware that executes software, firmware, or combinations thereof. The implementation can be as a computer program product, for example a computer program tangibly embodied in a non-transitory machine-readable storage device, for execution by, or to control the operation of, data processing apparatus, for example a computer, a programmable processor, or multiple computers.

Further, as shown in FIG. 12, the processor 1210 can also be connected to various interfaces via a system or an input/output (I/O) interface 1250 (e.g., USB connector, audio interface, FireWire, interface for connecting peripheral devices, etc.). The I/O interface 1250 can connect the processor to a camera 1270, coupled to the viewer device 10, that can be used for observing and recording samples using the viewer device 10. The camera 1270 can obtain images of an observation sample (not shown here) using the viewer device 10 and forward the images to the processor for processing or presenting to a user (not shown) on the display 1230 of the system 1200. Further, any data obtained from observing and recording experiments can be stored on a data storage 1240 of the system 1200.

The display 1230 can be any suitable display available in the art, for example a Liquid Crystal Display (LCD) or a light emitting diode (LED) display. The display can further be a touch screen display that can receive instructions from a user (e.g., instructions regarding observing and/or obtaining an image of an experiment).

The processor 1210 can also control the functions of the camera 1270 in response to instructions received from the main memory 1220 and the software application 1222. The software application 1222 can further include software applications that can store and process images obtained by the camera 1270 through the viewer device 10. Examples of such processing can include interpreting recorded images by analyzing the colors, shapes, sizes, or responses of features in the recorded images to generate digital information. The I/O interface 1250 can further be connected to other peripherals, such as one or more speakers for acoustic output, a microphone for acoustic input.

The processor 1210 can also be connected to a network interface 1260. The communications interface 1260 can provide the viewer device 10 with a connection to a communications network, such as the Internet. Transmission and reception of data, information, and instructions can occur over the communications network.

Further, although not shown, the viewer device 10 can be attached to a detection or sensing device, which can detect or sense various phenomena including, such as, light, fluorescence, or magnetism, or some combination thereof. Such non-camera based detection or sensing device(s) can be attached to device 10 to observe samples or monitor changes other than image changes discussed thus far. A viewer device according to the present teachings can be fabricated using any suitable material and manufacturing technique. For example, in some embodiments, the body of the viewer device can be formed of a suitable plastic using e.g., molding.

While the invention has been particularly shown and described with reference to specific illustrative embodiments, it should be understood that various changes in form and detail may be made without departing from the spirit and scope of the invention. Further, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

What is claimed is:

1. A viewer device comprising:
a housing providing a chamber for receiving a sample holder, said housing comprising:
a viewing window for observing a sample contained in said sample holder when the sample holder is disposed in said chamber; and
a port through which the sample holder can be introduced into, and removed from, said chamber.

2. The viewer of device of claim 1, further comprising a coupling mechanism for removably and replaceably coupling said housing to an observational device such that said observational device can generate data about the sample via said viewing window.

3. The viewer device of claim 2, wherein said data comprises image data.

4. The view device of claim 2, wherein said observational device is a portable device.

5. The viewer device of claim 2, wherein said observational device comprises a camera equipped device for obtaining any of a picture and video of said sample.

6. The viewer device of claim 5, wherein said camera-equipped device is configured for acquiring visible images of a sample. The viewer device of claim 5, wherein said camera-equipped device is configured for acquiring at least one of infrared or ultraviolet images of a sample.

8. The viewer device of claim 5, wherein said camera-equipped device comprises any of a smartphone, a portable computer, a laptop computer, or any device having an image acquisition mechanism included therein.

9. The viewer device of claim 1, wherein said coupling mechanism comprises any of a clip, a railing, one or more clamps, magnetic attachments, or vacuum cups, or pads.

10. The viewer device of claim 1, wherein said coupling mechanism is configured to allow aligning a camera of said camera-equipped device with said viewing window.

11. The viewer device of claim 10, wherein said coupling mechanism comprises at least one clip for removably and replaceably attaching the housing to the observational device, a railing to which the clip is movably coupled for adjusting the position of the viewer device relative to the observational device, and a knob coupled to the clip and the railing for securing the viewer device at a desired position along the railing.

12. The viewer device of claim 1, wherein said viewing window comprises a lens.

13. The viewer device of claim 1, further comprising a light source coupled to said housing for illuminating said sample holder when disposed in said chamber.

14. The viewer device of claim 1, wherein at least a portion of said sample holder is substantially transparent to visible radiation to allow said light to illuminate at least a portion of sample contained within the sample holder.

15. The viewer device of claim 14, further comprising a light diffuser optically coupled to said light source for diffusing the light emitted by the light source such that the diffused light illuminates the sample.

16. The viewer device of claim 13, wherein said light source comprises any of a light emitting diode (LED), a halogen lamp, a fluorescent, and a compact fluorescent light bulb.

17. The viewer device of claim 13, further comprising a color filter optically coupled to the light source for selecting a desired color for illuminating the sample.

18. The viewer device of claim 1, further comprising a locking mechanism coupled to said housing for engaging with at least a portion of the sample holder so as to inhibit movement of said sample holder in said chamber.

19. The viewer device of claim 18, wherein locking mechanism comprises a lip disposed proximate to said port for engaging with a proximal end of a sample holder disposed in said chamber so as to inhibit at least one of up-down movement and rotation of the sample holder.

20. The viewer device of claim 1, further comprising a mechanism coupled to said housing for manipulating said sample holder disposed in said chamber.

21. The viewer device of claim 20, wherein said mechanism for manipulating the sample holder is configured to cause any of physical shaking, rotation, spinning, and reversing orientation of said sample holder disposed in said chamber.

22. The viewer device of claim 20, wherein said mechanism for manipulating the sample holder comprises at least one spring disposed in said chamber for mechanical coupling to said sample such that pulling up the sample at least partially from the chamber and releasing it can cause agitation thereof.

23. The viewer device of claim 1, further comprising a heater coupled to said housing for heating a sample contained in said sample holder.

24. The viewer device of claim 1, further comprising a cooler coupled to said housing for cooling a sample contained in said sample holder.

25. The viewer device of claim 1, wherein said sample holder comprises any of a test tube, a filter, a slide, a line of continuously flowing fluid, an insect, a coin, a gem, a stamp, a hair, or any sample holder that allows light to reach the sample.

26. The viewer device of claim 1, wherein said sample holder comprises at least one fluidic channel for receiving the sample.

27. The viewer device of claim 1, wherein said chamber is configured for simultaneously receiving a plurality of sample holders.

28. The viewer device of claim 1, wherein said observational device comprises an analysis module for analyzing said data to generate information about the sample.

29. The viewer device of claim 28, wherein said information indicates a level of aggregation of at least one constituent of said sample.

30. The viewer device of claim 28, wherein said information indicates color of at least a portion of said sample.

31. A system for analyzing a sample, comprising:
a sample holder having at least one fluidic channel for receiving a sample;
an input port for introducing the sample to said fluidic channel; and
a viewer device, comprising:
a housing providing a chamber for receiving said sample holder, said housing comprising:
a viewing window for observing at least a portion of the sample flowing through said fluidic channel;
a port through which the sample holder can be introduced into, and removed from, said chamber; and
a coupling mechanism for removably and replaceably coupling said housing to an observational device such that said observational device can generate data via said viewing window about a sample flowing through said at least one fluidic channel.

32. The system of claim 31, wherein said observational device comprises a camera-equipped device.

33. A viewer device comprising:
a housing providing a chamber for receiving a sample holder, said housing comprising:
a viewing window for observing a sample contained in said sample holder when the sample holder is disposed in said chamber;
a port through which the sample holder can be introduced into, and removed from, said chamber; and
a camera coupled to said housing and configured to obtain images of said sample via said viewing window.

34. The viewer device of claim 33, wherein said camera is integrated within said housing.

35. A viewer device comprising:
a housing providing a chamber for receiving a sample holder, said housing comprising:
a viewing window for observing a sample contained in said sample holder when the sample holder is disposed in said chamber;
a port through which the sample holder can be introduced into, and removed from, said chamber; and
an observational device configured to generate data about the sample via said viewing window.
